(19) United States
(12) Patent Application Publication
(10) Pub. No.: US 2003/0191451 A1
(43) Pub. Date: Oct. 9, 2003

(54) Reinforced catheter system

(76) Inventor: Kevin Gilmartin, Kingston, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/117,695

(22) Filed: Apr. 5, 2002

(51) Int. Cl. A61M 25/00

(52) U.S. Cl. 604/527

(57) ABSTRACT

Herein is disclosed a reinforced catheter system that includes a reinforcement layer including a helical strand, wherein the helical strand has a stiffness of about 30% to about 100% greater than the stiffness of other helical strands used for reinforcing the catheter. The catheter may include a single reinforcement layer or multiple layers. Advantageously, the catheter provides for enhanced kink resistance for navigation of small and/or tortuous vascular channels, and provides enhanced hoop strength for protecting catheter integrity when the device is subjected to increased pressure within its lumen, for example, during the delivery of intravascular agents.

### FIELD OF THE INVENTION

The present invention relates to medical tubes. More particularly, the present invention relates to medical tubes that may be used in catheters.

### BACKGROUND OF THE INVENTION

A number of medical applications advantageously use catheters that are adapted for advancement through small and/or tortuous blood vessels to reach an anatomical location where an intravascular agent may be delivered. Agents useful as intravascular agents may include, but are not limited to, contrast agents, therapeutic agents, diagnostic agents, and embolic agents. When the desired anatomical location has been reached by the catheter, the intravascular agent may be delivered thereto, typically by the application of pressure through a channel of the catheter or by mechanical manipulation. The pressure required for this delivery exerts radial stress on the catheter through which the agent is delivered. It is desirable, therefore, that the catheter be constructed to resist this stress so that the stress does not deform or damage the catheter or adversely affect the delivery of the agent.

Other handling characteristics for catheters have been identified that facilitate their advancement through small and/or tortuous blood vessels. It is desirable, for example, that a catheter have a certain torsional rigidity so that torque can be effectively transmitted from the proximal manipulable end of the catheter to the distal tip. As a catheter is advanced, the medical practitioner typically rotates the proximal end with the expectation that that rotational movement will be effectively imparted to the distal end. An appropriate amount of torsional rigidity facilitates this effective transmission. It is also desirable that a catheter have a certain longitudinal rigidity along at least a portion of it so that it can be effectively pushed forward through narrow blood vessels. Additionally, however, it is desirable that the distal end of the catheter have a certain degree of flexibility so that it can follow the course of narrow or tortuous blood vessels and avoid damaging them. Moreover, resistance to kinking is desirable when a catheter is advanced through narrow or tortuous channels; the presence of kinking interferes with the handling properties of the catheter and furthermore compromises the lumen through which the intravascular agent is to be delivered.

There remains a need in the art for in medical tube adapted for use in a catheter which provides the combination of handling characteristics desirable for reaching anatomic sites via tortuous and/or narrow vascular channels and further desirable for delivering intravascular agents thereto.

### SUMMARY OF THE INVENTION

Disclosed herein are medical tubes for use in a catheter with desirable handling characteristics, in particular handling characteristics suitable for the navigation of small and/or tortuous blood vessels. These handling characteristics may include a flexible distal portion, kink resistance, torque transferring ability, longitudinal strength or rigidity, and hoop strength. One embodiment provides a catheter having a kink-resistant medical tube that has a longitudinal axis directable through a blood vessel. The medical tube may be resilient and flexible. Disposed within the wall of the tube is a filamentous tubular support member that includes two sets of filaments, a first set of filaments disposed helically in the first direction and a second set of filaments disposed helically in a second direction that may be braided with the first set of filaments. The first set of filaments may have a plurality of filaments with a first stiffness and may further have at least one filament with a second stiffness which is about 30 percent to about 100 percent greater than the first stiffness. The filamentous tubular support member may include metallic or plastic filaments. The majority of the filaments of the first set of filaments may have the first stiffness, and 75 percent to 80 percent or more of the first set of filaments may have the second stiffness. The filaments in the first set may be different in cross-sectional shape from the filaments of the second set, and furthermore the filaments of the first set may have a different cross-sectional shape from the at least one filament in that set having the greater stiffness. The at least one filament, also referred to as the “reinforcing filament,” may have, furthermore, a rounded contour and it may be formed from a metallic or a plastic material or any other material familiar to skilled artisans.

Also disclosed herein are tubular sheaths for use as a component of a catheter with desirable handling characteristics, in particular handling characteristics suitable for the navigation of small and/or tortuous blood vessels. One embodiment may provide a catheter having a tubular sheath that has a first reinforcement layer and a second reinforcement layer. These reinforcement layers may be adjacent to each other or separated from each other by any other layer or layers. These reinforcement layers may be found in conjunction with other layers lining the tubular sheath or coating it. The first reinforcement layer may be external to the second reinforcement layer or internal to it. A third reinforcement layer may be added to the tubular sheath and placed in any relation to the other reinforcement layers. The third reinforcement layer may comprise braided filamentous strands. The first reinforcement layer may include a first set of helically disposed filamentous strands and the second reinforcement layer may include a second set of helically disposed filamentous strands, the first set and the second set crossing each other in a relationship of helical rotation and helical counterrotation. Within the first set of helically disposed filamentous strands, a majority of strands will have a first stiffness and further will have at least one strand with a second stiffness of about 30 percent to 100 percent greater than the first stiffness. Strands in the reinforcement layers may be made from metallic, plastic or any other materials. The at least one strand may also be made from metallic, plastic or any other materials, those materials being the same as or different than the materials used to form the other strands within the first or the second reinforcement layer. In one embodiment, about 12 percent to about 25 percent of the helically disposed filamentous strands of the first set are replaced with the at least one strand.

Further disclosed herein are layered catheter assemblies with desirable handling characteristics, in particular handling characteristics suitable for the navigation of small and/or tortuous blood vessels. One embodiment may provide a catheter having two layers, a first layer and a second layer. The first layer may include a tubular reinforcement layer having a set of helical strands embedded therein, each helical strand of the set having a substantially similar stiffness. The second layer may include a counterrotating helically wound strand having a stiffness about 30 percent to about 100 percent greater than the stiffness of the strands in the first layer. The first layer may be external to or internal to the second layer. The helically wound strand may be made from metallic materials or plastic materials or any other suitable material. In one embodiment, a third layer may be included that is external to the first layer and the second layer, or that is internal to the first layer and the second layer. The third layer may include helical strands as well, or may be braided.

Also disclosed herein are tubular sheaths useful in reinforced catheter systems that include a braided reinforcement layer and a helical reinforcement layer that has a set of helically wound strands with a first stiffness and at least one strand having a second stiffness that is about 30% to about 100% greater than the first stiffness. The braided reinforcement layer may be disposed external to the helical reinforcement layer or internal to the helical reinforcement layer.

The catheter reinforcement systems disclosed herein may offer certain attributes desirable for facilitating the direction of a catheter through narrow and/or tortuous vessels. These attributes include, but are not limited to, kink resistance, stretch resistance, and increased ability to transmit torque. These desirable features may be obtained by using different size filaments within a braid matrix to supply additional mechanical advantage. Herein is disclosed a system wherein a standard braid that is added to a catheter shaft is combined with a helical coil to provide increased kink resistance, as compared to the braid alone, without stiffening the shaft as significantly. This combination offers advantages over using braid alone in amounts sufficient to increase the shaft's ability to transmit torque so as to navigate narrow passages, to increase the shaft's ability to resist ruptures from fluidic forces within the catheter lumen, and to decrease the shaft's ability to stretch or distort.

These and other features of the depicted embodiments will be more fully understood by referring to the following detailed description and the accompanying drawings, wherein like numbers reference like features.

### BRIEF DESCRIPTION OF THE FIGURES

The features of the systems disclosed herein believed to be novel are set forth with particularity in the following claims. However, these systems, together with further objects and advantages thereof, may be best understood by reference to the following description of illustrative embodiments taken in conjunction with the accompanying drawings, wherein:

FIG. 1 shows a longitudinal perspective view of an embodiment of a medical tube suitable for use as a component of a catheter.

FIGS. 2A and 2B depict schematically a longitudinal projection and a cross-sectional view of an embodiment of a filamentous tubular support member.

FIGS. 3A and B illustrate a longitudinal perspective view of an embodiment of a tubular sheath suitable for use as a component of a reinforced catheter, and a cross-sectional view of same.

FIGS. 3C and 3D each show a longitudinal view of an embodiment of a tubular sheath suitable for use as a component of a reinforced catheter.

FIG. 4 depicts schematically an embodiment of a catheter.

### DETAILED DESCRIPTION OF FIGURES

Reference will now be made in detail to certain illustrative embodiments. Each example is provided for the purpose of explanation and is not meant as a limitation. For example, features illustrated or described as part of one embodiment can be used on another embodiment to yield yet another embodiment. It is intended that the scope of the appended claims include such modifications and variations.

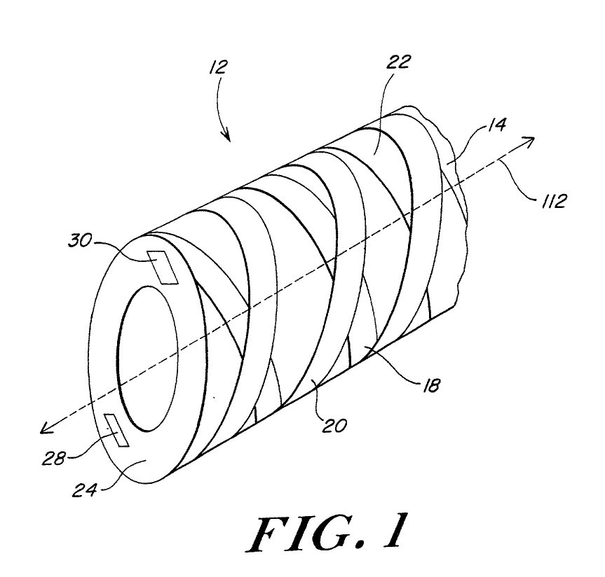

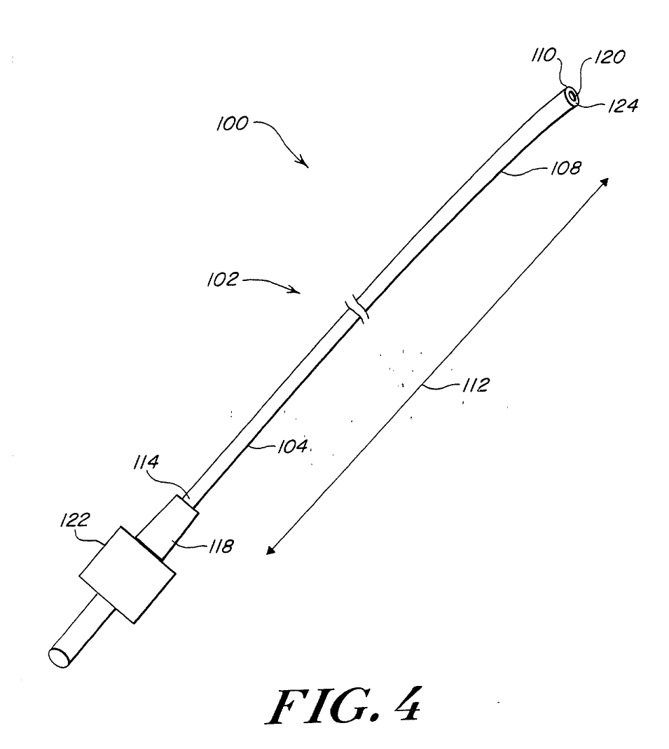

FIG. 1 illustrates a tube 12 having a longitudinal axis 112 that is suitable for use as a component of a catheter. Features of a catheter are described in more detail with reference to FIG. 4. Desirably, the tube 12 is flexible and kink-resistant or resilient. Embedded in the wall 24 of the tube 12 is a filamentous tubular support member 14. In certain embodiments, the filamentous tubular support member 14 extends along the entire length of the tube, terminating, for example just proximal to the distal tip of a catheter, as may be appreciated by referring to FIG. 4. This termination point is desirable so that the materials forming the filamentous tubular support member 14 do not protrude out the tip or affect its properties or capacities for fluid delivery. In other embodiments, reinforcement from the filamentous tubular support member 14 will not be advantageous over a proximal portion of the catheter, particularly when more rigid polymers are used for fabricating this portion so that additional kink resistance is not required. The filamentous tubular support member 14 includes a first set of filaments 18 disposed helically in a first direction, and a second set of filaments 20 disposed helically in a second direction. In certain embodiments, the filamentous tubular support member 14 is formed from a braiding of the first set of filaments 18 and the second set of filaments 20. The first set of filaments 18 and the second set of filaments 20 may be arranged in counterrotating helical directions, or in other intersecting or crossing or non-crossing directions as will be understood by those of ordinary skill in the art. The braiding arrangement disposes the first set of filaments 18 and the second set of filaments 20 in relation to each other at a preselected strand angle. As understood herein, the strand or braid angle is measured between the strand or filament and the longitudinal axis 112 of the tube 12. While in certain embodiments, a narrow strand angle may be preferred to increase bending flexibility, in other embodiments, a more open strand angle may be preferred to decrease bending flexibility. Similarly, the braiding arrangement disposes a given number of filaments along a specified length of the catheter, providing a braid with a given pic count. As is understood by skilled artisans, the braid angle is dependent upon the pic count. As the pic count increases, the braid angle may approach 90 degrees. In certain embodiments, both the pic count and the strand angle may have different values along the length of the catheter, so as to modify the catheter properties for a particular catheter region. Variations of pic count and strand angle to effect this goal will be familiar to practitioners in the art. In one embodiment, for example, a soft and flexible distal tip may be advantageous for the negotiation of tortuous blood vessels and the like. In this exemplary embodiment, the strand angle or the pic count or both could be modified at the distal end of the catheter so as to provide this property. Concomitantly, the reinforcement provided by the at least one strand 22, also termed the reinforcing strand 22, may maintain hoop strength at the distal end sufficient to minimize tendencies for kinking and for radial distortion of the catheter when it is used, for example, to deliver intravascular agents under pressure to a site distal to the distal end of the catheter. While the Figure shows only one reinforcing strand 22, it is understood that a plurality of reinforcing strands 22 may be provided in accordance with the disclosure herein.

Among the first set of filaments 18 is a plurality of filaments with a first stiffness. Among the first set of filaments 18 is also at least one filament 22 having a second stiffness. The second stiffness, i.e., the stiffness of the reinforcing filament 22 is about 30 percent to about 100 percent greater than the first stiffness. In certain embodiments, the majority of filaments (i.e., greater than 50 percent) among the first set of filaments 18 will have the first stiffness as described above. Advantageously, 75 percent to 80 percent of the filaments in the first set of filaments 18 will have the first stiffness while, respectively, 20 percent to 25 percent of the filaments will have the second stiffness, i.e., the stiffness of the aforesaid reinforcing filament 22, that is about 30 percent to about 100 percent greater than the first stiffness.

As used herein, the term “stiffness” relates to the property of a filament that contributes to the hoop strength of the catheter rather than to the longitudinal strength, rigidity or flexibility. The stiffness of the filaments comprising the filamentous tubular support member 14 may be varied by varying the material composition of a filament or filaments or the cross-sectional area of a filament or filaments. This can be achieved by varying the width of a filament, the thickness of a filament, or the diameter of a filament. In one embodiment, it is especially desirable that the width of the at least one filament 22 be increased to enhance stiffness (i.e., hoop strength). Not to be bound by theory, it is understood that increasing the amount of coverage of the tube 12 by a wide, flat reinforcing filament 22 may increase stiffness (i.e., hoop strength) and be advantageous for preventing kinking of the tube 12.

As shown in FIG. 1, the filaments comprising the filamentous tubular support member 14 are disposed within the wall 24 of the tube 12. As illustrated by this Figure, the first set of filaments 18 has a different cross-sectional shape 30 than the cross-sectional shape 28 of the second set of filaments 20. To increase the stiffness of the filament 22, its width, its diameter, its cross-sectional shape and/or cross-sectional area or its material construction may be varied. In one embodiment, the reinforcing filament 22 may have a cross-sectional shape that differs from the cross-sectional shape of other filaments in the first set of filaments 18. The cross-sectional shape of the reinforcing filament 22 may be rounded, square, rectangular or substantially flat, as may be the shapes of the other filaments forming the filamentous tubular support member 14. Combinations of shapes may be used for the different filaments, so that the first set of filaments 18 may, for example, have a different shape than the second set of filaments 20, and vice versa. As used herein, the term “rounded” includes those shapes that are oval, ovoid or round, as well as those with rounded edges. In certain embodiments, the filamentous tubular support member 14 may comprise metallic filaments. In other embodiments, the filamentous tubular support member 14 may comprise plastic filaments. Specifically, the at least one filament 22 may be formed from a metallic material or a plastic material, which material may be the same as or different than the materials used for other filaments.

A variety of materials may be used for the reinforcing filament 22 and for the other filaments of the filamentous tubular support member 14, as will be appreciated by those of ordinary skill in the art. In one embodiment, the first and second set of filaments may comprise polymers such as Kevlar, nylon, polyurethane or polypropylene. The reinforcing filament 22 may be fabricated using a heavier gauge or stiffer strand of a polymer, or may be fabricated from a metal, such as a stainless steel or a Nitinol alloy or a precious metal. In certain embodiments, a radio-opaque marker may be added at the end of the catheter or may be incorporated in one of the filaments or formed by one of the filaments if the material used for its formation is radioopaque.

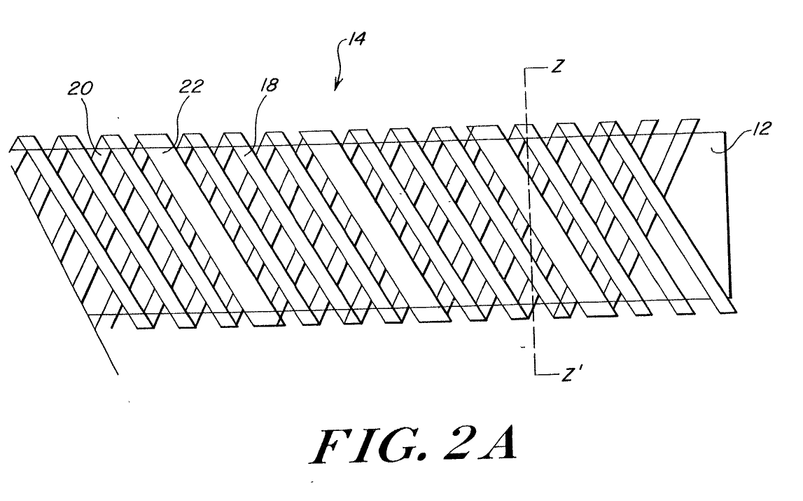

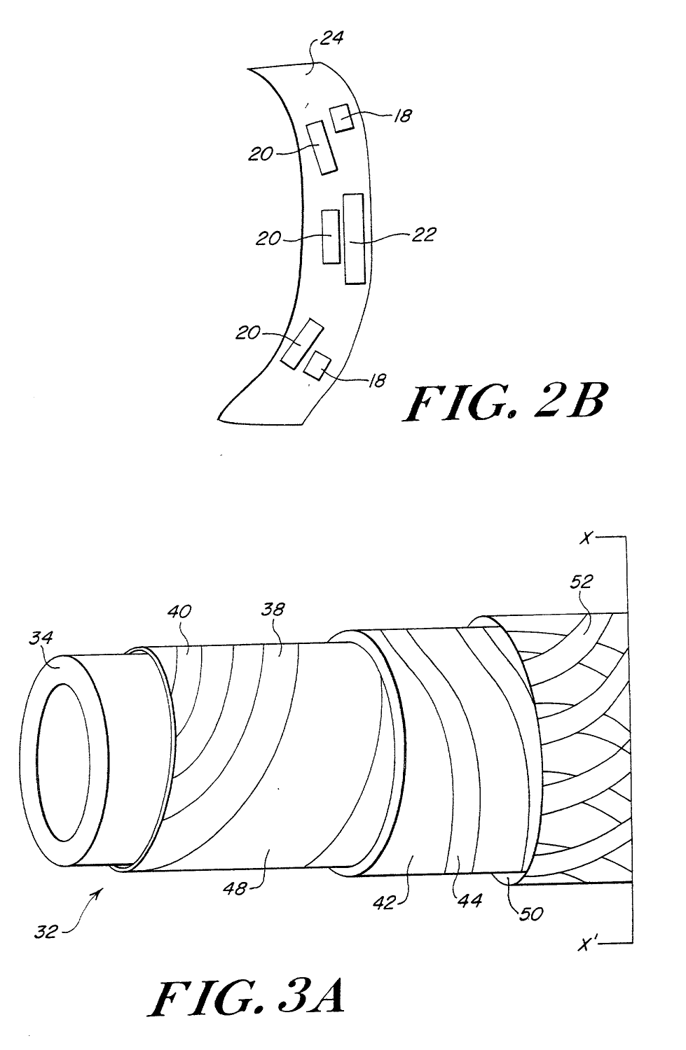

FIG. 2A shows diagrammatically an embodiment of a filamentous tubular support member 14 as may be disposed within the wall of a tube 12 useful for forming a catheter. As depicted herein, there is a first set of filaments 18 disposed helically in a first direction which may be braided with a second set of filaments 20 disposed in a second helical direction. In one embodiment, for example, a standard braiding pattern maybe used (for example 16 filaments, 100 pics per inch, 8 over/8 under braid pattern with 0.0005 in.×0.003 in. #304 stainless steel) with one to four filaments substituted with a material which is about 30% to 100% stiffer than the other filaments wrapped in the same direction of the braid. Increasing the stiffness may be accomplished by increasing the width or the thickness of the replacement filament, or by increasing the cross-sectional area, or by altering the material so that a substance imparting greater rigidity or strength is used. As described above, at least one filament 22, a reinforcing filament 22, of the first set of filament 18 has a stiffness that is about 30 percent to about 100 percent greater than the stiffness of the other filaments in the first set 18. In certain embodiments, this filamentous wire tubular support member 14 may be used as a reinforcing coil within a catheter. The dotted line z-z′ indicates the site of a cross section that will be depicted in FIG. 2B.

FIG. 2B shows in more detail a cross section through a tubular support member 14 within the wall 24 of a tube useful for forming a catheter. As shown in this Figure, filaments from the first set of filaments 18 are disposed within the wall 24 and have a first cross-sectional area that corresponds to a particular stiffness. Certain filaments from the second set of filaments 20 are also disposed within the wall 24 and may have a second cross-sectional area that corresponds to a different stiffness. Among the first set of filaments 18 is at least one filament 22 having a stiffness that is about 30 percent to about 100 percent greater than the stiffness of the other filaments in the first set of filaments 18, as previously described. Other arrangements of the first set of filaments 18 and the second set of filaments 20 within the wall 24 may be readily envisioned by practitioners of ordinary skill in the art.

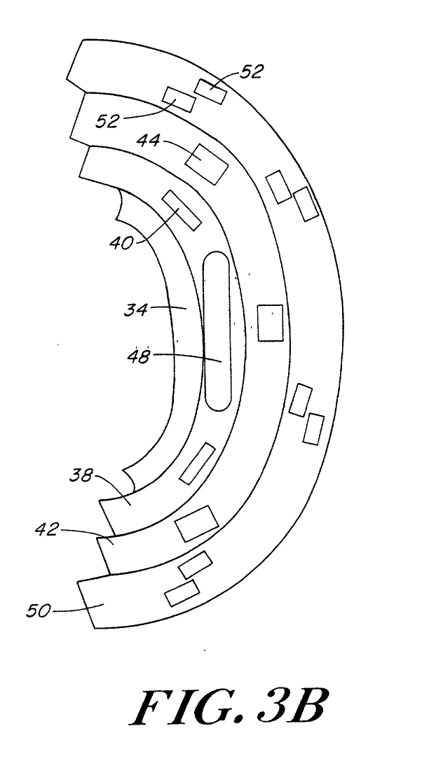

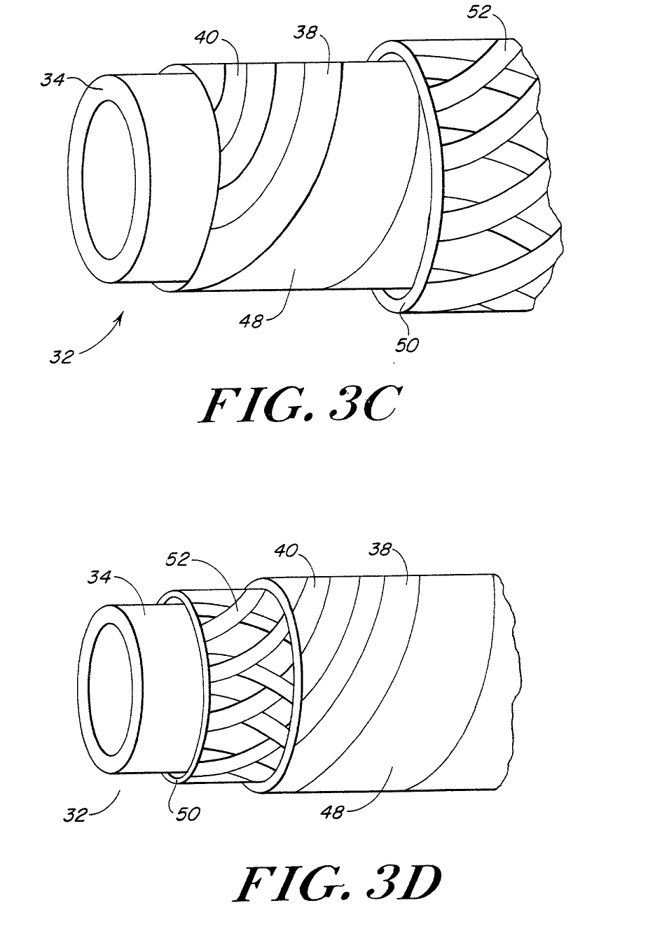

FIGS. 3A-D, described in more detail below, provide illustrations of arrangements of reinforcing layers for tubular sheath constructions useful in forming reinforced catheters. The reinforcing layers may use reinforcing filaments as have been described previously. The reinforcing filaments may be formed in the previously described shapes and formed using the previously described materials.

FIG. 3A shows an embodiment of a tubular sheath 32 suitable for use as a component of a reinforced catheter. The tubular sheath 32 as depicted in this Figure includes an inner tube 34 or inner layer made from a biocompatible material. The inner layer 34 may be omitted entirely in certain embodiments of the tubular sheath 32 or may be replaced with a coating of a biocompatible material surrounding a lumen of the tubular sheath 32. Substances useful for the inner layer 34 are familiar in the art and may include, for example, polymers such as Teflon or polyethylene. Other layers forming the tubular sheath may be formed of these polymers or of others such as PolyetherBlockAmide, polyvinyl chloride or polyurethane. An outermost coating (not shown) provided for the tubular sheath may be formed of a polymer such as nylon, polyvinyl chloride or PolyetherBlockAmide. As will be understood in the art, a variety of materials may be envisioned that are appropriate for the formation of the different sheath layers, and skilled artisans will be readily able to select the appropriate material without undue experimentation.

As shown in FIG. 3A, a first reinforcement layer 38 may be disposed external to the optional inner tube 34. This first reinforcement layer 38 bears a first set of helical strands 40 included among which is at least one strand 48 with a stiffness of about 30 percent to about 100 percent greater than the stiffness of the other strands in the first set of helical strands 40. Overlying this first reinforcement layer 38 may be a second reinforcement layer 42 bearing a second set of helical strands 44 rotating in a direction opposite to that of the first set of helical strands 40. Optionally, a braided reinforcement layer 50 may be provided, comprising, for example, braided filamentous strands 52. While this figure shows the braided reinforcement layer 50 external to the other two reinforcement layers 38 and 42, it is understood that the position of the braided reinforcement layer may be placed internal to one or both of the two reinforcement layers 38 and 42. The line at x-x′ indicates where a cross-section shown in more detail in FIG. 3B is taken.

FIG. 3B shows in more detail a cross section of the tubular sheath 32 depicted in FIG. 3A. As this Figure shows, an inner layer 34 may be present adjacent to a lumen (not shown) of the tubular sheath. External to the inner layer 34 may be a first reinforcement layer 38 which bears a first set of helical strands 40 wound in a first helical direction. The first set of helical strands 40 includes at least one strand 48, a reinforcing strand, with a stiffness of about 30 percent to about 100 percent greater than the stiffness of the other strands in the first set of helical strands 40. External to this first reinforcement layer 38 may be a second reinforcement layer 42 bearing a second set of helical strands 44 wound in a second helical direction, desirably in a direction that is the opposite of the first helical direction. External to the second reinforcement layer 42 may be an optional braided reinforcement layer 50 bearing, for example, braided filamentous strands 52 or helical strands. Although FIG. 3B shows the third layer external to the second layer and the second layer external to the first layer, it will be understood by practitioners of ordinary skill in the art that the arrangement of the layers can be varied in accordance with well-known principles of catheter design. For example, the optional braided reinforcement layer 50 may be placed internal to the first layer 38 and the second layer 42. This arrangement may offer particular advantages. Alternatively, the optional braided reinforcement layer 50 may be placed internal to only one of the other reinforcement layers. Similarly, the first layer 38 with its at least one reinforcing strand 48 may be disposed internal or external to any of the other layers described herein. Other arrangements can be readily envisioned by skilled artisans.

FIG. 3C depicts another embodiment of a tubular sheath 32 suitable for use with a reinforced catheter. As this Figure shows, an inner layer 34 may be present adjacent to a lumen of the tubular sheath. External to the inner layer 34 is disposed a helical reinforcement layer 38 that bears a set of helical strands 40 wound in a first helical direction. The set of helical strands 40 includes at least one strand 48 with a stiffness of about 30 percent to about 100 percent greater than the stiffness of the other strands in the first set of helical strands 40. External to this helical reinforcement layer 38 may be a braided reinforcement layer 50 bearing braided strands 52 of wire or of some other reinforcing material such as plastic. Although FIG. 3C shows only two reinforcement layers, it will be understood by practitioners of ordinary skill in the art that additional layers, for reinforcement or for any other purpose, may be added to form a complete catheter assembly, in accordance with well-known principles of catheter design. Furthermore, although FIG. 3C shows the two reinforcement layers adjacent to each other, additional layers may be interposed between them.

With reference to FIG. 3D, another arrangement of reinforcement layers for a tubular sheath 32 suitable for a reinforced catheter may be appreciated. In the depicted embodiment, an inner layer 34 is shown, external to which a braided reinforcement layer 50 is illustrated bearing braided strands 52 of wire or of another reinforcing material familiar to skilled artisans. Plastics and metallics are both suitable for use in this layer. As described above, this inner layer 34 is optional and may be substituted with other biocompatible materials familiar in the art. A helical reinforcement layer 38 is shown external to the braided reinforcement layer bearing a set of helical strands 40 wound in a first helical direction. The set of helical strands 40 includes at least one strand 48 with a stiffness of about 30 percent to about 100 percent greater than the stiffness of the other strands in the first set of helical strands 40. Although FIG. 3D shows only two reinforcement layers, it will be understood by practitioners of ordinary skill in the art that additional layers, for reinforcement or for any other purpose, may be added to form a complete catheter assembly, in accordance with well-known principles of catheter design. Furthermore, although FIG. 3D shows the two reinforcement layers adjacent to each other, additional layers may be interposed between them.

FIG. 4 illustrates certain features of an embodiment of an intravascular catheter system such as a reinforced catheter as disclosed herein. Catheter 100 includes a shaft 102 having a longitudinal axis 112. The shaft 102 typically has a proximal region 104 and a distal region 108. In certain embodiments, the shaft may have a length between 80 and 150 cm, and may have a diameter of 3 Fr., though other dimensions for catheter construction will be familiar to those of ordinary skill in the art. The catheter 100 further has a proximal end 114 and a distal tip 110. A lumen orifice 120 is shown schematically along the distal face 124 of the distal tip 110. However, it will be understood that the orifice of the lumen 120 need not exit through a distal face 124 of the distal tip 110 but may be located anywhere along the distal tip 110 or indeed along the distal region 108 as indicated. The lumen interior to the catheter 100 is not visible in this figure. The lumen orifice 120 represents the distal end of the lumen (not visible). The lumen extends proximally from the distal orifice 120 through the catheter to a manifold 122 or similar proximal structure which includes the fittings (not shown) for a fluid source such as a syringe (not shown). The fluid source permits, for example, pressurized installation of fluid into the manifold and thence into the lumen of the catheter 100. The lumen accordingly provides fluid communication between the interior of the manifold 122 and the lumen orifice 120, through which fluid path may be injected diagnostic or therapeutic fluids such as an intravascular agent to a desired anatomic target site using conventional techniques. As depicted in this embodiment, a strain reliever 118 may be provided between the manifold 122 and a proximal region 104 of the catheter 100 in order to reduce the tendency of the shaft 102 to kink at the proximal end 114. While FIG. 4 illustrates features of conventional catheter design that would be suitable for use with the catheter reinforcement system disclosed herein, it will be understood by skilled practitioners that other catheter designs and other features thereof may be substituted for the depicted features to provide other suitable intravascular catheter constructions.

Equivalents

Those skilled in the art will be able to ascertain many equivalents to the specific embodiments described herein. The embodiments depicted and described herein are considered in all respects to be illustrative and not restrictive, the scope of the invention being indicated by the appended claims rather than by the foregoing description. All variations that come within the meaning and range of equivalency of the following claims therefore are intended to be embraced thereby.

I claim:

1. A catheter comprising:
a medical tube having a longitudinal axis; and
a filamentous tubular support member disposed within a wall of the tube,
the filamentous tubular support member having
a first set of filaments disposed helically in a first direction and braided with a second set of filaments disposed helically in a second direction,
the first set of filaments having a plurality of filaments with a first stiffness and further having at least one filament with a second stiffness about 30% to about 100% greater than the first stiffness.

2. The catheter of claim 1, wherein the filamentous tubular support member comprises metallic filaments.

3. The catheter of claim 1, wherein the filamentous tubular support member comprises plastic filaments.

4. The catheter of claim 1, wherein the first set of filaments has a majority of filaments with the first stiffness.

5. The catheter of claim 1, wherein the plurality of filaments has a first cross-sectional shape and the at least one filament has a second cross-sectional shape different from the first cross-sectional shape.

6. The catheter of claim 1, wherein the at least one filament has a rounded contour.

7. The catheter of claim 1, wherein the at least one filament comprises a metallic material.

8. The catheter of claim 1, wherein the at least one filament comprises a plastic material.

9. A catheter comprising:
a tubular sheath having a first reinforcement layer including a first set of helically disposed filamentous strands and having a second reinforcement layer including a second set of helically disposed filamentous strands, the first set and the second set crossing each other in a relationship of helical rotation and helical counterrotation,
the first set having a majority of strands with a first stiffness and further having at least one strand with a second stiffness of about 30%-100% greater than the first stiffness.

10. The catheter of claim 9, wherein the first reinforcement layer is adjacent to the second reinforcement layer.

11. The catheter of claim 10, wherein the first reinforcement layer is external to the second reinforcement layer.

12. The catheter of claim 9, wherein the at least one strand comprises a metallic material.

13. The catheter of claim 9, wherein the at least one strand comprises a plastic material.

14. The catheter of claim 9, wherein the at least one strand is rounded.

15. The catheter of claim 9 further comprising a third reinforcement layer of braided filamentous strands.

16. The catheter of claim 15, wherein the third reinforcement layer is disposed external to the first reinforcement layer.

17. The catheter of claim 16, wherein the third reinforcement layer is disposed external to the second reinforcement layer.

18. The catheter of claim 9, wherein one in four of the helically disposed filamentous strands of the first set is replaced with the at least one strand.

19. The catheter of claim 9, wherein between about 12% and about 25% of the helically disposed filamentous strands of the first set are replaced with the at least one strand.

20. A catheter comprising:
a first layer and a second layer,
the first layer comprising a tubular reinforcement layer having a set of helical strands embedded therein, each helical strand of the set having a substantially similar stiffness, and
the second layer comprising a helically wound strand having a stiffness about 30% to about 100% greater than the substantially similar stiffness.

21. The catheter of claim 20, wherein the second layer is external to the first layer.

22. The catheter of claim 20, wherein the first layer is external to the second layer.

23. The catheter of claim 20, wherein the helically wound strand comprises a metallic material.

24. The catheter of claim 20, wherein the helically wound strand comprises a plastic material.

25. The catheter of claim 20, wherein the helically wound strand is rounded.

26. The catheter of claim 20, further comprising a third layer including helical strands.

27. The catheter of claim 26, wherein the third layer is internal to the first layer and the second layer.

28. A reinforced catheter, comprising
a braided reinforcement layer, and
a helical reinforcement layer having a set of helically wound strands, wherein a first strand of the helically wound strands has a first stiffness and a second strand of the helically wound strands has a second stiffness, the second stiffness being about 30% to about 100% greater than the first stiffness.

29. The reinforced catheter of claim 28, wherein the helical reinforcement layer is external to the braided reinforcement layer.

30. The reinforced catheter of claim 28, wherein the helical reinforcement layer is internal to the braided reinforcement layer.

31. The reinforced catheter of claim 28, wherein the second strand comprises a metallic material

32. The reinforced catheter of claim 28, wherein the second strand comprises a plastic material.

33. The reinforced catheter of claim 28, wherein the second strand is rounded.
